(19) United States
(12) Patent Application Publication
(10) Pub. No.: US 2020/0323940 A1
(43) Pub. Date: Oct. 15, 2020

(54) DIETARY SUPPLEMENTS AND USE THEREOF ON MENSES

(71) Applicants: Clarisse LE COURT, PLENEUF VAL ANDRE (FR); CLARIPHARM, SAINT ALBAN (FR)

(72) Inventor: Clarisse LE COURT, PLENEUF VAL ANDRE (FR)

(73) Assignees: CLARIPHARM, SAINT ALBAN (FR); Clarisse LE COURT, SAINT ALBAN (FR)

(21) Appl. No.: 16/956,312

(22) PCT Filed: Dec. 21, 2018
(86) PCT No.: PCT/FR2018/053514

§ 371 Date: Jun. 19, 2020

(30) Foreign Application Priority Data
Dec. 21, 2017 (FR) 1762870

(51) Int. Cl. A61K 36/73; A23L 33/00; A61K 33/26; A61K 47/22; A61K 47/46

(52) U.S. Cl. CPC A61K 36/73; A23L 33/30; A61K 47/46; A61K 47/22; A61K 33/26

(57) ABSTRACT

The invention relates to dietary supplements for use in the field of menstruation, in particular painful and heavy menstruation. The invention relates to a composition based on potentilla and iron.

The present invention relates to the field of dietary supplements.

The invention more particularly relates to dietary supplements for the use thereof in the field of menstruation and the associated discomfort.

In the field of menstruation, and particularly painful and heavy menstruation, said menstruation results in discomfort for more than 75% of women. This discomfort manifests itself in strong abdominal cramps, a sensation of fatigue and weakness, anemia, diarrhea, headaches, nausea and vomiting.

In order to alleviate this discomfort, various solutions are based on passing on traditional remedies that have evolved over time. For example, the use of poultices involving various techniques for application and various compositions. Nonetheless, all these techniques and compositions require a relatively long, and relatively impractical, or even awkward, application time.

Another solution that is provided is in the form of heat, of the bath or hot water bottle type. This technique relaxes the abdominal muscles by dilation, which may reduce pain. Nonetheless, this technique is accompanied by an intensification of the blood flow in the hours following the application of heat, which is unpleasant.

Another contemplated solution has been that of monitoring food, caffeine, alcohol, fats and carbohydrates. Furthermore, monitoring food is prohibitive and demanding. Moreover, an increase in carbohydrates, in particular glucose, leads to the production of insulin, causing the activation of pro-inflammatory prostaglandins implicated in the pain and heaviness of menstruation.

Finally, another contemplated solution involves the use of different treatments. A first treatment may be the use of medication which can cause numerous undesirable side effects. Moreover, this medication may also be accompanied by contraindications. Other treatments recommend the use of a contraceptive. However, a hormone-based oral contraceptive, for example, needs to be taken regularly and is not very well suited to alleviating painful and heavy menstruation.

In order to overcome these disadvantages, the invention proposes dietary supplements for the use thereof in the field of menstruation.

The invention more particularly relates to a dietary supplement composition comprising potentilla and iron. In this composition, the percentage by weight of potentilla is between 20% and 55%, while the percentage by weight of iron is between 0.008% and 0.09%.

Advantageously, potentilla helps to maintain a good level of comfort throughout the menstrual cycle. In addition, potentilla has anti-inflammatory and pain-reducing properties.

Advantageously, iron contributes to the normal formation of red blood cells and hemoglobin and to the compensation of iron lost due to menstruation. In addition, iron reduces fatigue and weakness.

According to a first feature of the invention, a daily dose of potentilla weighs between 0.25 g to 7 g, preferably a daily dose of potentilla weighs between 4 to 6 g.

According to a second feature of the invention, a daily dose of iron weighs between 1 mg and 14 mg, preferably a daily dose of iron weighs between 2 mg and 7 mg.

According to a third feature of the invention, the composition comprises ginger at a percentage by weight of between 3% and 25%. In particular, a daily dose of ginger weighs between 0.25 g and 4 g, preferably a daily dose of ginger weighs between 1 g and 2 g.

According to a fourth feature of the invention, the composition comprises vitamin E or vitamin K at a percentage of less than 0.1%.

More specifically, the composition can comprise vitamin E at a percentage by weight of between 0.008% and 0.06%, preferably a daily dose of vitamin E weighs between 1 mg and 15 mg.

In addition, the composition can also comprise vitamin K at a percentage by weight of between 0.0001% and 0.0005%, preferably a daily dose of vitamin K weighs between 10 μg and 75 μg.

According to a fifth feature of the invention, the composition can comprise yarrow at a percentage by weight of between 15% and 70%. However, a daily dose of yarrow weighs between 0.15 g and 15 g.

According to a sixth feature of the invention, the composition comprises a cinnamon extract at a percentage by weight of between 10% and 25%. According to the invention, a daily dose of the cinnamon extract weighs between 0.5 g and 2 g.

According to a seventh feature of the invention, the composition can comprise a pomegranate extract at a percentage by weight of between 15% to 45%, and preferably the pomegranate extract has a percentage by weight of between 20% to 25%. According to the invention, a daily dose of the pomegranate extract weighs between 1 g and 4 g, and preferably a daily dose of the pomegranate extract weighs between 1.5 g and 2.5 g.

The invention also relates to a method for limiting heavy menstruation, comprising the regular, preferably daily, administration of a composition according to the invention.

The administration is preferably carried out over a period ranging from one to several days, more preferably 3 days, before the start of menstruation, up to one to several days, more preferably 2 days, after the start of menstruation.

The invention will be described in further detail by the description of non-limiting embodiments.

The present invention relates to the field of dietary supplements. The invention more particularly relates to dietary supplements for the use thereof in the field of menstruation.

The invention is particularly applied, in a non-limiting manner, in the alleviation of painful and heavy menstruation, respectively.

The dietary supplement composition according to the invention comprises potentilla. Potentilla is a perennial plant from arctic and temperate regions, belonging to the genus Potentilla and the Rosaceae family. A species favored for the composition according to the invention is Potentilla anserina. Furthermore, the aerial part of potentilla is preferably used for the production of the dietary supplement composition according to the invention. In particular, an extract of the aerial parts concentrated to 4/1 is used.

Potentilla helps to maintain a good level of comfort before and during a menstrual cycle. Furthermore, potentilla regulates the menstrual cycle. Moreover, potentilla has anti-inflammatory properties and helps to reduce pain. The aerial part of Potentilla anserina is preferred and yields good results.

In light of these effects, the dietary supplement composition according to the invention comprises a percentage by weight of potentilla of between 20% and 55%; preferably, the percentage by weight of potentilla is between 25% and 35%.

These percentages by weight preferably correspond, for a daily dose, to a potentilla weight of between 0.25 g and 7 g. The potentilla weight of a daily dose is preferably between 4 g and 6 g. A daily dose corresponds to the amount to be administered per day.

This potentilla weight corresponds to an herb equivalent. Concentrated commercial extracts can be used. Once the potentilla has been reconstituted, the corresponding weight is chosen from the preferred ranges of the present application.

In a particularly preferred variant, the potentilla weight is preferably between 4 g and 6 g. These ranges make it possible to draw sufficient benefit from the effects of the potentilla. This weight is preferably less than 7 g. Indeed, above 7 g per daily dose, it is acknowledged that potentilla is liable to cause digestive disorders.

The dietary supplement composition according to the invention comprises iron. Iron is one of the essential trace elements of the body. Iron is an essential constituent of hemoglobin and myoglobin as well as several enzymes and cytochromes. Iron is also present in the circulation, bound to transferrin, and in the form of ferritin reserves, predominantly in the liver. During menstruation, losses of iron are increased and may even lead to deficiencies.

Iron contributes to compensating for the iron lost during menstruation, particularly when said menstruation is heavy. Furthermore, the supply of iron enables the normal formation of red blood cells and hemoglobin that are newly synthesized in order to compensate for the losses. Moreover, iron helps to reduce fatigue and weakness.

In this context, the dietary supplement composition according to the invention can comprise iron at a percentage by weight of between 0.008% and 0.09%. The iron preferably has a percentage by weight of between 0.02% and 0.006%.

In this example, these percentages by weight correspond, for a daily dose, to an iron weight of between 1 mg and 15 mg, the iron weight is preferably between 2 mg and 7 mg. This is particularly the weight of a daily dose.

The ranges make it possible to draw sufficient benefit from the effects of the iron in the context of the invention. A weight of between 2 mg and 7 mg yields excellent results. Above 15 mg, the composition exceeds the recommended daily allowances for a menstruating woman.

It should be noted that the iron may be used in the form of ferric sodium EDTA complex. This type of complex is commercially available, for example, under the name Ferrazone™. The complex makes it possible to make the iron bioavailable.

The dietary supplement composition according to the invention comprises at least one additional component in the form of excipient. The excipient enables the stability of the composition, facilitates the biological transport thereof, influences palatability and enables a specific mode of administration and pharmaceutical form. According to a preferred embodiment of the invention, the dietary supplement composition is in the form of tablets.

Surprisingly, the applicant realized that iron and potentilla, when combined, mutually contribute to the generation of a beneficial effect on the discomfort of menstruation. More specifically, iron facilitates the formation of red blood cells, while potentilla produces an anti-inflammatory effect which limits pain.

According to one embodiment of the invention, the dietary supplement composition can comprise ginger.

Ginger is a plant with an aromatic rhizome, native to Asia and belonging to the Zingiberaceae family. A species favored for the dietary supplement composition suitable for alleviating heavy menstruation according to the invention is Zingiber officinale Roscoe. Furthermore, the roots of ginger are preferably used to produce the composition.

Ginger contributes to physical wellbeing. Furthermore, ginger comprises anti-inflammatory agents and has an inhibitory effect on prostaglandin synthesis, which makes it possible to reduce hypermenorrhea. Indeed, hypermenorrhea is associated with an increase in the prostaglandin concentration.

However, the reduction in hypermenorrhea produced by the ginger is combined with the beneficial effects of the potentilla and the iron in order to alleviate the discomfort of menstruation.

In this sense, the dietary supplement composition can comprise a percentage by weight of ginger of between 3% and 25%. The percentage by weight of ginger is preferably between 9% and 15%.

In particular, these percentages by weight correspond, for a daily dose of ginger, to a weight of between 0.25 g and 4 g. The ginger weight is preferably between 1 g and 2 g. This weight is an equivalent in ginger, and in particular in ginger rhizome.

The dietary supplement composition according to the invention can also comprise vitamins. In particular, the dietary supplement composition can comprise vitamin K at a percentage by weight of less than 0.1%.

The effects of vitamin K will now be examined. It is also possible to more preferably use vitamin K1. Vitamin K is a vitamin derived from quinones. It is an enzyme cofactor which is also involved in the maturation of certain coagulation factors.

Thus, vitamin K helps to generate normal blood coagulation. However, it has been demonstrated that a vitamin K deficiency may lead to hemorrhages which increase the blood flow during menstruation and excessive menstruation.

Consequently, a dietary supplement composition comprising vitamin K more particularly targets the alleviation of heavy menstruation.

Advantageously, vitamin K ensures normal blood coagulation which makes it possible to limit blood losses during menstruation. Thus, when vitamin K is combined with potentilla and iron, the beneficial effects of these compounds cooperate so as to alleviate the discomfort of heavy menstruation.

Continuing with the aim of providing a dietary supplement composition to alleviate heavy menstruation, the composition can comprise vitamin K at a percentage by weight of between 0.0001% and 0.0005%. Preferably, the vitamin K has a percentage by weight of between 0.00025% and 0.00035%.

More specifically, these percentages by weight correspond, for a daily dose, to a vitamin K weight of between 10 μg and 75 μg. The vitamin K weight is preferably between 12 μg and 35 μg. Such a weight gives good results. A daily supply of more than 75 μg exceeds the recommended allowances.

The dietary supplement composition can also comprise a pomegranate extract. Pomegranate, Punica granatum, is a fruit from the Punicaceae family and is found on the pomegranate tree. In this example, a pomegranate extract is used that is derived from dried pomegranate flowers. Nonetheless, it is also possible to use the fruit or the leaves of the pomegranate tree, or the seeds.

Pomegranate contains flavanols, ellagitannins, flavonoids and anthocyanosides. These chemical compounds afford pomegranate hypotensive and anti-cholesterol effects. Moreover, a study showed that pomegranate helps to reduce the duration of bleeding, thus also reducing menstrual losses. Pomegranate thereby makes it possible to increase the concentration of hemoglobin following a menstrual cycle.

In this context, a pomegranate extract may be introduced together with vitamin K, in order to form a dietary supplement composition which aims to alleviate heavy menstruation. Indeed, the reduction in the duration of bleeding helps to alleviate the phenomenon of heavy menstruation.

To this end, the dietary supplement composition can comprise a pomegranate extract at a percentage by weight of between 15% to 45%. The percentage by weight of pomegranate is preferably between 20% to 25%.

These percentages by weight preferably correspond, for a daily dose, to a pomegranate extract weight of between 1 g and 4 g. The pomegranate extract weight of a daily dose is preferably between 1.5 g and 2.5 g. Such a weight yields significant results, while a daily supply of more than 4 g exceeds the recommended allowances.

According to another possibility of the invention, the dietary supplement composition may comprise a cinnamon extract. Cinnamon, Cinnamonium cassia, from the Lauraceae family, is found on a tree called the Chinese cinnamon tree. In this example, an aqueous extract of Chinese cinnamon bark has been used. This aqueous extract has a 4/1 ratio.

Cinnamon comprises compounds of the proanthocyanidin and cinnamaldehyde type, which are particularly renowned for their antioxidant property. Cinnamon is also known for its antibacterial and pain-relieving properties, etc. Moreover, a study recently showed that cinnamon reduces blood losses, digestive disorders (pain, nausea, vomiting) and also pain during the menstruation period.

In light of these beneficial effects on heavy menstruation, a cinnamon extract can be added to a dietary supplement composition seeking to alleviate heavy menstruation. Indeed, the reduction in blood losses, digestive disorders and pain participates, together with the effects of the other components of this composition, in alleviating the phenomenon of heavy menstruation.

To this end, the dietary supplement composition can comprise a cinnamon extract at a percentage by weight of between 10% and 25%. The cinnamon extract preferably has a percentage by weight of between 12% and 17%.

It should be noted that these percentages by weight correspond to a daily dose of between 0.5 g and 2 g of cinnamon extract. The cinnamon extract weight of a daily dose is preferably between 1 g and 1.5 g.

Such a weight yields significant results, while a daily supply of more than 2 g exceeds the recommended allowances.

It is also possible to include vitamin E in the dietary supplement composition at a percentage by weight of less than 0.1%. Vitamin E is a liposoluble vitamin derived from the chromanol nucleus. It is an antioxidant that reduces oxidative stress. It prevents the excessive aggregation of platelets responsible for thrombosis, and has a protective action on red blood cells.

Vitamin E interacts with other vitamins and hormones, in particular progesterone which has a relaxant effect on the uterine muscles and reduces the risk of hemorrhage. Furthermore, vitamin E modulates inflammatory processes by virtue of its action on the prostaglandins involved in uterine contractions and hence pain. Consequently, vitamin E helps to reduce premenstrual and menstrual pain.

Consequently, adding vitamin E to a dietary supplement composition particularly allows menstrual pain to be alleviated.

Advantageously, the reduction in premenstrual and menstrual pain which is promoted by vitamin E helps, in collaboration with the effects of the iron and the potentilla, to alleviate the pain associated with painful menstruation.

It should be noted that the vitamin E can be in the form of alpha-tocopherol and more particularly alpha-tocopherol succinate. Succinate makes it possible to increase the preservation of the alpha-tocopherol and also the absorption thereof.

With this in mind, the dietary supplement composition comprises vitamin E at a percentage by weight of between 0.008% and 0.06%. The vitamin E preferably has a percentage by weight of between 0.01% and 0.03%.

For a daily dose, these percentages by weight correspond to a vitamin E weight of between 1 mg and 12 mg. The weight of a daily dose is preferably between 2 mg and 6 mg. Such a weight yields significant results. Conversely, a weight per dose of more than 12 mg would exceed the recommended daily allowances.

In addition, the dietary supplement composition may also comprise yarrow.

Yarrow is a spreading root plant belonging to the Asteraceae family. According to the invention, it is preferable for Achillea millefolium to be used. By way of an example, the tops or upper parts of yarrow can be used in the form of powder in the context of the invention.

Yarrow, by virtue of its antispasmodic and anti-inflammatory properties, helps to soothe abdominal cramps by interacting, for example, with progesterones. Consequently, yarrow enables a reduction in premenstrual and menstrual pain. In this sense, it is possible to integrate yarrow into a dietary supplement composition in order to treat painful menstruation.

In this context, the dietary supplement composition can comprise yarrow at a percentage by weight of between 15% and 70%. The percentage of yarrow is preferably between 35% and 55%.

These percentages by weight preferably correspond, for a daily dose, to a yarrow weight of between 0.15 g and 15 g. The yarrow weight of a daily dose is preferably between 9 g and 13 g. These weight ranges yield good results for alleviating discomfort associated with painful menstruation.

The invention further relates to the use of the compositions described above, in particular for alleviating painful menstruation or limiting heavy menstruation.

More particularly, the above compounds are administered below the recommended daily doses, in particular less than 14 mg of active iron per day, less than 7 g of potentilla equivalent per day.

### EXAMPLES

In order to illustrate the dietary supplement composition according to the invention, the applicant provides embodiments of the invention hereafter.

Firstly, examples 1 to 5 of tables 1 to 3 below correspond to embodiments, in accordance with the invention, of a dietary supplement composition for alleviating painful menstruation.

TABLE 1

| | Example 1 | | Example 2 | |
|---|---|---|---|---|
| | Dose/day | % | Dose/day | % |
| Compounds | (grams) | by weight | (grams) | by weight |
| Potentilla | 0.5 | 22.21 | 6 | 37.47 |
| anserina | | | | |
| Iron | 0.001 | 0.04 | 0.014 | 0.09 |
| Pomegranate | 1 | 44.42 | 4 | 24.98 |
| extract | | | | |
| Cinnamon | 0.5 | 22.21 | 2 | 12.49 |
| extract | | | | |
| Ginger | 0.25 | 11.11 | 4 | 24.98 |
| Vitamin K | 0.00001 | 0.0004 | 0.000075 | 0.0005 |
| Totals | 2.25101 | 100.00 | 16.014075 | 100.00 |

TABLE 2

| | Example 3 | | Example 4 | |
|---|---|---|---|---|
| | Dose/day | % | Dose/day | % |
| Compounds | (grams) | by weight | (grams) | by weight |
| Potentilla | 4 | 53.32 | 6 | 49.97 |
| anserina | | | | |
| Iron | 0.002 | 0.03 | 0.007 | 0.06 |
| Pomegranate | 1.5 | 19.99 | 2.5 | 20.82 |
| extract | | | | |
| Cinnamon | 1 | 13.33 | 1.5 | 12.49 |
| extract | | | | |
| Ginger | 1 | 13.33 | 2 | 16.66 |
| Vitamin K | 0.00001125 | 0.0001 | 0.0000375 | 0.0003 |
| Totals | 7.502 | 100 | 12.007 | 100 |

Example 5 in table 3 below is a dietary supplement composition suitable for alleviating heavy menstruation. The dose is administered in the form of tablets.

TABLE 3

| | | PER | % | ACTUAL ACTIVE | |
|---|---|---|---|---|---|
| | PER | UNIT | ACTIVE | INGREDIENTS/D | |
| COMPONENTS | 100 G | (mg) | INGREDIENTS | (mg) | OVERDOSAGE |
| Ferrazone | 2.02% | 19.320 | 13.00% | 10 | |
| Vitamin K1 at 1% | 0.03% | 0.325 | 1.00% | 0.013 | 10% |
| Rhizome ginger | 3.95% | 37.500 | | | |
| DE 5/1 | | | | | |
| Potentilla DE 4/1 | 26.32% | 250.00 | | | |
| Microcrystalline | 42.52% | 403.913 | | | |
| cellulose 102 | | | | | |
| Heavy calcium | 24.21% | 230.00 | 37.24% | 342.609 | |
| carbonate DC 95 MD | | | | | |
| Tricafos 200-7 | 0.95% | 9.000 | | | |
| Carboxymethyl | 0.00% | 0.030 | | | |
| cellulose | | | | | |

Although the composition of example 5 is for alleviating heavy menstruation, it does not comprise either cinnamon extract or pomegranate extract.

Microcrystalline cellulose 102, heavy calcium carbonate DC 95 MD, Tricafos 200-7 and carboxymethyl cellulose are commercially available agents for manufacturing tablets.

The regular, preferably daily, for example, tablet form administration of the dietary supplement composition suitable for alleviating heavy menstruation helps to limit heavy menstruation. Preferably, the dosage regimen is more than 3 and preferably less than 5 units per day. A dosage regimen of 4 units per day makes it possible to remain within the recommended nutritional values for the majority of the elements of the composition (see table 3, “actual active ingredients per day” column). Each unit weighs 950 mg without film coating.

The tablets preferably have a longitudinal shape in order to facilitate ingestion. This may be a cylindrical shape with rounded bases.

More preferably, administration may be carried out over a period of 5 consecutive days ranging from 3 days before menstruation to 2 days after the start of menstruation. It is possible to contemplate a variant in which administration begins when symptoms appear and/or ends when symptoms disappear.

Examples 6 to 10, illustrated in tables 4 to 6 below, correspond to embodiments, according to the invention, of a dietary supplement composition for alleviating heavy menstruation.

TABLE 4

| | Example 6 | | Example 7 | |
|---|---|---|---|---|
| | Dose/day | % | Dose/day | % |
| Compounds | (grams) | by weight | (grams) | by weight |
| Potentilla | 0.5 | 39.94 | 7 | 26.90 |
| anserina | | | | |
| Iron | 0.001 | 0.08 | 0.014 | 0.05 |
| Yarrow | 0.5 | 39.94 | 15 | 57.63 |
| Vitamin E | 0.001 | 0.08 | 0.012 | 0.05 |
| Ginger | 0.25 | 19.97 | 4 | 15.37 |
| Totals | 1.252 | 100 | 26.026 | 100 |

TABLE 5

| | Example 8 | | Example 9 | |
|---|---|---|---|---|
| | Dose/day | % | Dose/day | % |
| Compounds | (grams) | by weight | (grams) | by weight |
| Potentilla | 4 | 24.99 | 6 | 28.55 |
| anserina | | | | |
| Iron | 0.002 | 0.01 | 0.007 | 0.03 |
| Yarrow | 11 | 68.73 | 13 | 61.87 |
| Vitamin E | 0.0018 | 0.01 | 0.006 | 0.03 |
| Ginger | 1 | 6.25 | 2 | 9.52 |
| Totals | 16.0038 | 100 | 21.013 | 100 |

### Example 10

Example 10 is a dietary supplement composition suitable for alleviating painful menstruation. The dose is administered in the form of tablets.

TABLE 6

| | | PER | % | ACTUAL ACTIVE | |
|---|---|---|---|---|---|
| | PER | UNIT | ACTIVE | INGREDIENTS/D | |
| COMPONENTS | 100 G | (mg) | INGREDIENTS | (mg) | OVERDOSAGE |
| Ferrazone | 2.02% | 19.320 | 13.00% | 10 | |
| Vitamin E succinate | 0.39% | 3.717 | 77.36% | 11.5 | 15% |
| Yarrow | 15.79% | 150.000 | | | |
| Potentilla DE 4/1 | 26.32% | 250.00 | | | |
| Microcrystalline | 30.32% | 288.022 | | | |
| cellulose 102 | | | | | |
| Heavy calcium | 24.21% | 230.000 | 37.24% | 342.609 | |
| carbonate DC 95 MD | | | | | |
| Tricafos 200-7 | 0.95% | 9.000 | | | |
| Carboxymethyl | 0.00% | 0.030 | | | |
| cellulose | | | | | |

The dietary supplement composition of example 10 is suitable for alleviating painful menstruation.

The dosage regimen is preferably similar to the dosage regimen of example 5, in particular, for example, involving taking four units per day. Each unit weighs 950 mg without film coating.

More preferably, administration may be carried out over a period of 5 consecutive days ranging from 3 days before menstruation to 2 days after the start of menstruation. It is possible to contemplate a variant in which the administration begins when symptoms appear and/or ends when symptoms disappear.

1. A dietary supplement composition comprising, as percentage by weight:
between 20% and 55% potentilla; and
between 0.008% and 0.09% iron.

2. The composition according to claim 1, wherein a daily dose of potentilla weighs between 0.25 g to 7 g, preferably a daily dose of potentilla weighs between 4 to 6 g.

3. The composition according to claim 1, wherein a daily dose of iron weighs between 1 mg and 14 mg, preferably a daily dose of iron weighs between 2 mg and 7 mg.

4. The composition according to claim 1, comprising ginger at a percentage by weight of between 3% and 25%.

5. The composition according to claim 4, wherein a daily dose of ginger weighs between 0.25 g and 4 g, preferably a daily dose of ginger weighs between 1 g and 2 g.

6. The composition according to claim 1, comprising vitamin E or vitamin K at a percentage of less than 0.1%.

7. The composition according to claim 6, comprising vitamin E at a percentage by weight of between 0.008% and 0.06%, preferably a daily dose of vitamin E weighs between 1 mg and 15 mg.

8. The composition according to claim 6, comprising vitamin K at a percentage by weight of between 0.0001% and 0.0005%, preferably a daily dose of vitamin K weighs between 10 μg and 75 μg.

9. The composition according to claim 1, comprising yarrow at a percentage by weight of between 15% and 70%.

10. The composition according to claim 9, wherein a daily dose of yarrow weighs between 0.15 g and 15 g.

11. The composition according to claim 1, comprising a cinnamon extract at a percentage by weight of between 10% and 25%.

12. The composition according to claim 11, wherein a daily dose of the cinnamon extract weighs between 0.5 g and 2 g.

13. The composition according to claim 1, comprising a pomegranate extract at a percentage by weight of between 15% to 45%, and preferably the pomegranate extract has a percentage by weight of between 20% to 25%.

14. The composition according to claim 13, wherein a daily dose of the pomegranate extract weighs between 1 g and 4 g, and preferably a daily dose of the pomegranate extract weighs between 1.5 g and 2.5 g.

15. The composition according to claim 7, for the use thereof in the treatment of painful menstruation.

16. The composition according to claim 2, wherein a daily dose of iron weighs between 1 mg and 14 mg, preferably a daily dose of iron weighs between 2 mg and 7 mg.

17. The composition according to claim 2, comprising ginger at a percentage by weight of between 3% and 25%.

18. The composition according to claim 3, comprising ginger at a percentage by weight of between 3% and 25%.

19. The composition according to claim 2, comprising vitamin E or vitamin K at a percentage of less than 0.1%.

20. The composition according to claim 3, comprising vitamin E or vitamin K at a percentage of less than 0.1%.
